(19) United States
(12) Patent Application Publication
(10) Pub. No.: US 2007/0064983 A1
(43) Pub. Date: Mar. 22, 2007

(54) Method for automatically detecting nasal tumor

(76) Inventors: Wen-Chen Huang, Kaohsiung (TW); Chun-Pin Chang, Rende Township (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/227,259

(22) Filed: Sep. 16, 2005

(51) Int. Cl. G06K 9/00; A61B 5/05

(52) U.S. Cl. 382/128; 600/410

(57) ABSTRACT

The present invention discloses a method for automatically detecting a nasal tumor from the MR (magnetic resonance) images. First, the pixels that have specific trends and are affected by contrast agents with specific level will be filtered according to the developing coefficient and control coefficient of grey prediction. Then the tumor area would be detected by using Fuzzy C-means clustering technique to distinguish the differences between normal tissue and tumor. Owing to the work of grey prediction, calculation in the Fuzzy C-means clustering technique can be dramatically reduced and the result of tumor detection is enhanced.

### BACKGROUND OF THE INVENTION

1. Field of the Invention

The present invention relates to a method for automatically detecting a nasal tumor, and particularly to a method for automatically detecting nasal tumor by grey prediction and Fuzzy C-means clustering technique.

2. Related Prior Arts

Inverted Papilloma (IP) is a benign epithelial tumor that arises from the mucous membrane of the nasal cavity and paranasal sinuses, most commonly the lateral nasal wall in the region of the middle meatus. It is a relatively common neoplasm of the nasal cavity. Surgery is needed for good outcome; and the importance of diagnosing recurrent IPs lies in the fact that a high recurrence rate (15%-78%) and associated epithelial malignant transformation may be coexistence in 5.5%-27% of cases. Patients typically present with nasal obstruction, epistaxis, or nasal discharge. Owing to high recurrent rates and associated malignant transformation of IPs, it is very important to evaluate the efficacy of conventional or pharmacokinetic MRI (magnetic resonance imaging) in the differentiation between recurrent tumor and post-treatment changes in the follow-up of patients with IPs after operation.

MR images have the characteristics of noninvasive, radiation-free, high-resolution, sensitive to the tumor tissue, and could be viewed from different angles to observe the abnormal structure and the relationship among its neighborhood. MRI becomes a very important diagnosis tool for clinic inspection. In addition, the further advancement of dynamic MRI techniques to pharmacokinetic examination allows a more detailed characterization of contrast medium enhancement in tissue.

Dynamic MRI is one of the major nasal tumor detection tools and is widely used by radiologists. Huang et al. presents a system to detect and enhance the tumor region by computing the relative intensity difference between consecutive MR images after using contrast agent, referring to “Recurrent Nasal Tumor Detection by Dynamic MRI,” IEEE Engineering in Medicine and Biology, pp. 100-105, July/August, 1999. They apply a relative signal increase (RSI) model to recognize the recurrent nasal tumor from dynamic MR images.

However, the process of locating the region of interest (ROI) of tumor requires the identification by the users in the first place. If the priori knowledge is false then the consequent process would not be correct.

In order to resolve such a problem, a new fully automatic tumor detection technique is proposed.

### SUMMARY OF THE INVENTION

The purpose of this research is to detect and enhance the tumor region in DMRI automatically by using grey prediction and Fuzzy C-means.

The method for automatically detecting a nasal tumor of the present invention comprises steps of: (a) roughly segmenting at least two MR (magnetic resonance) images by grey prediction to locate candidate tumor regions; and (b) refmedly segmenting said MR images of step (a) by Fuzzy C-means clustering to filter a possible tumor region from normal regions.

Preferably, the MR images of step (a) are previously transformed into a grey level format, more preferably without header information. The MR images typically have a width of 256 pixels and a height of 256 pixels, and the images can be transformed into 256 grey levels in each pixel thereof.

Preferably, the corresponding points of the MR images are previously matched with each other, more preferably by the phase correlation process and the function minimization process.

In the above step (a), the images are preferably segmented according to developing coefficient and control coefficient of grey prediction.

More merits and features are illustrated in the following description accompanied with the drawings.

### BRIEF DESCRIPTION OF THE DRAWINGS

FIG. 1 is the flowchart of the proposed system.

FIG. 2 shows Dynamic MRI with 15 time frames after image registration.

FIG. 3 shows the intensities change of the tumor region.

FIG. 4 shows the rough segmentation of possible tumor area.

FIG. 5 shows the possible tumor region after rough segmentation.

FIG. 6 shows the results after performing the proposed automatic tumor detection algorithm.

### DETAILED DESCRIPTION OF THE PREFERRED EMBODIMENTS

There are two assumptions for a preferred embodiment in accordance with the present invention:
(1) The intensities change more rapidly among those tumor regions than the normal areas; and
(2) The regions in the head MR image can be divided into two categories: tumor region and normal region.

Under the first assumption, grey prediction is used to differentiate between the tumor and normal regions. As described in ‘Deng, Ju-Long, “Introduction to Grey System Theory,” J. Grey System, vol. 1, no. 1, 1-24, 1989’, the grey prediction uses a finite number of numeric values with specific characteristic to predict the needed values. In the grey prediction model, two operators are used as the basic tools, that is accumulated generating operation (AGO) and inverse accumulated generating operation (IAGO). AGO is applied on the original series to make it more regular. Therefore, we can use the differential equation as prediction model to approximate such regularity. IAGO help us to get the needed values from the series calculated by prediction model, eventually. In the dynamic MRI, the intensity has different changing property between the tumor region and normal region. After the grey prediction procedure, the differences between the tumor and the normal region discriminate the possible tumor region from MRI.

Under the second assumption, the Fuzzy C-means (FCM) clustering is used to distinguish possible tumor locations. FCM uses the principles of fuzzy sets to generate a membership distribution function while minimizing a fuzzy entropy measure, as mentioned in “L. O. Hall, A. M. Bensaid, L. P. Clarke, R. P. Velthuizen, M. S. Silbiger, and J. C. Bezdek. A comparison of neural network and fuzzy clustering techniques in segmenting magnetic resonance images of the brain. IEEE Transactions on Neural Networks, 3(5):672-682, 1992.”

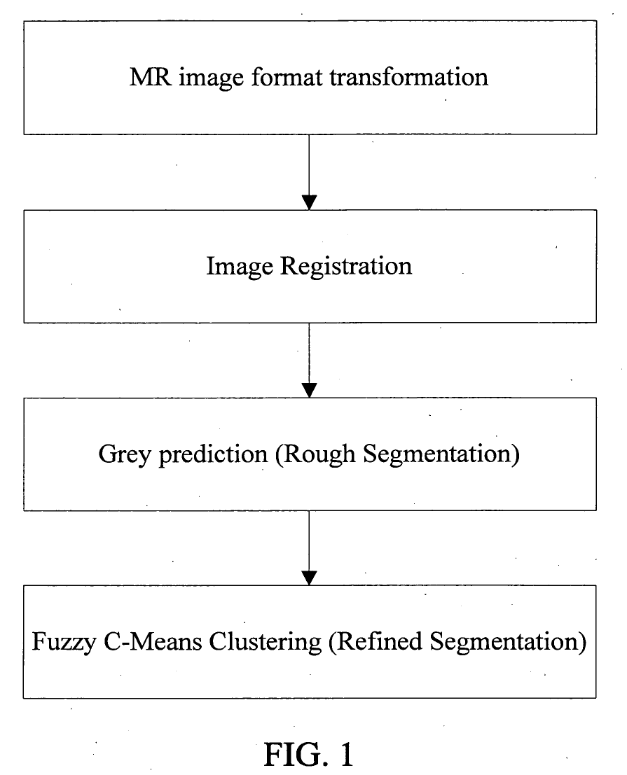

FIG. 1 is the flowchart of the preferred embodiment, in which there are two stages: preprocessing and segmentation. In the preprocessing stage, two steps are performed: MR image format transformation and image registration. The segmentation algorithm is divided into two steps: grey prediction (rough segmentation) and Fuzzy C-means clustering (refined segmentation). Detailed procedures are illustrated as follows.

(1) The Preprocessing Stage

(1a) Transforming Formats of MR Images

A total of nine dynamic FSE (fast spin-echo) images were obtained at 0, 5, 30, 60, 90, 120, 150, 180, and 300 seconds after bolus Gd-DTPA injection. The original MR image contains 138,976 bytes with the image header of 7,904 bytes. The image width is 256 pixels and the image height is 256 pixels. The number of bits in an uncompressed pixel is 16 and this MR image is uncompressed normal rectangular image. Notice that the intensity value of each pixel is specified by the reverse sequence of high_byte and low_byte. The intensity value of each pixel is equal to the summation of the high_byte value and the multiplication of the low_byte value by 256. That is, I=low_byte×256+high_byte, where I is the intensity value of each pixel. For most computer display device, the maximum displayed grey level is 256. The original 2-byte intensity value is converted into 1-byte intensity value. The final image format become raw image of 256×256 without the header information and each pixel has 256 grey levels. After the image file format transformation, the MR image can be displayed on the computer screen.

(1b) Matching Corresponding Points of the MR Images

Because of the movement of patients, the sequences of serial MR images taken from consecutive time are not corresponding to each other in the same pixel position. The images need to be aligned with one another so that any type of analysis can base on the correct data. Therefore, matching the corresponding points from different images become very important. It would be tedious and imprecise by selecting the corresponding points by clicking the mouse on the screen from the user. Over the years, a technique called “Image Registration” have been developed to deal with the matching corresponding points from two or more pictures taken, from different sensors, at different times, or from different viewpoints. Two methods, “phase correlation” and “function minimization”, are selected for dealing with the motion correction problem. Basically, the problem is tried to solve the motion parameters m₁ to m₈ in equation (1).

$$[ \begin{matrix}{x}^{1} \\ {y}^{1} \\ {w}^{1}\end{matrix} ] = [ \begin{matrix}{m}_{1} & {m}_{2} & {m}_{3} \\ {m}_{4} & {m}_{5} & {m}_{6} \\ {m}_{7} & {m}_{8} & 1\end{matrix} ] [ \begin{matrix}x \\ y \\ w\end{matrix} ] \qquad ( 1 )$$

wherein m₃ and m₆ are related to the translation of x and y position; m₁, m₂, m₄, and m₅ are related to the rotation parameters. If it is the rigid motion, then m₁=cos θ, m₂=−sin θ, m₄=sin θ, and m₅=cos θ; m₇ and m₈ are related to the scaling factors because of the projective distortion.

The “phase correlation” technique was proposed by Kuglin and Hines in 1975. This method is suited for large displacements between the two images and provides good initial guesses for matched image pairs.

This technique estimates the 2-D translation between a pair of images by taking 2-D Fourier Transforms of each image, computing the phase difference at each frequency, performing an inverse Fourier Transform, and searching for a peak in the magnitude image.

After obtaining the initial guesses of the matched image pairs, the “function minimization” technique is used to minimize the discrepancy in intensities between pairs of images after applying the motion transformation. In other words, this technique minimizes the sum of the squared intensity errors

$$E = {\sum }_{i} {[ {I}^{} ( {x}_{i}^{1} , {y}_{i}^{1} ) - I ( {x}_{i} , {y}_{i} ) ]}^{2} = {\sum }_{i} {e}_{i}^{2} \qquad ( 2 )$$

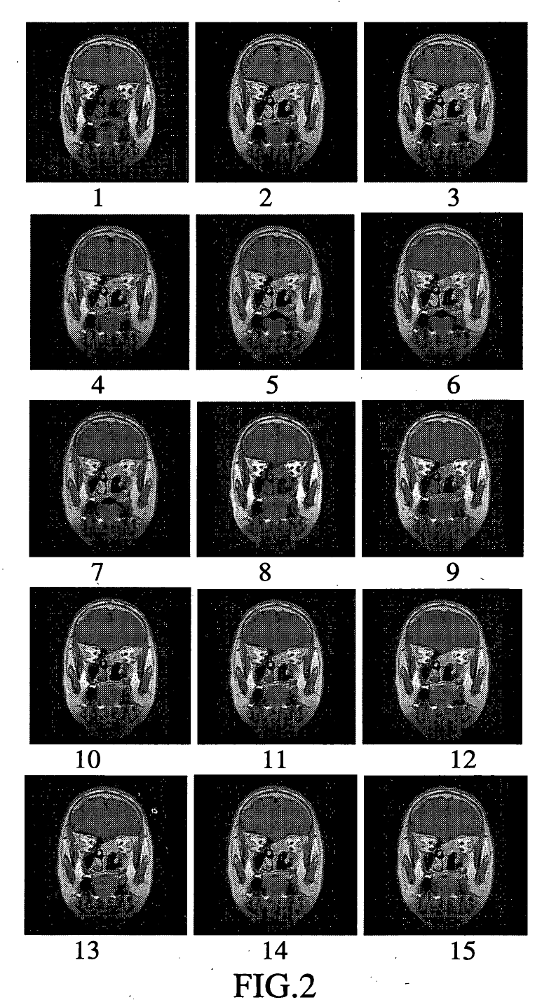

over all corresponding pairs of pixels i which are inside both images I(x,y) and I′(x′,y′). To perform the minimization, this algorithm requires the computation of the partial derivatives of eᵢ with respect to the unknown motion parameters {m₀Λ m₇ }. FIG. 2 shows Dynamic MRI with 15 time frames after image registration.
(2) The Segmenting Stage
(2a) Roughly Segmenting the Images by Grey Prediction

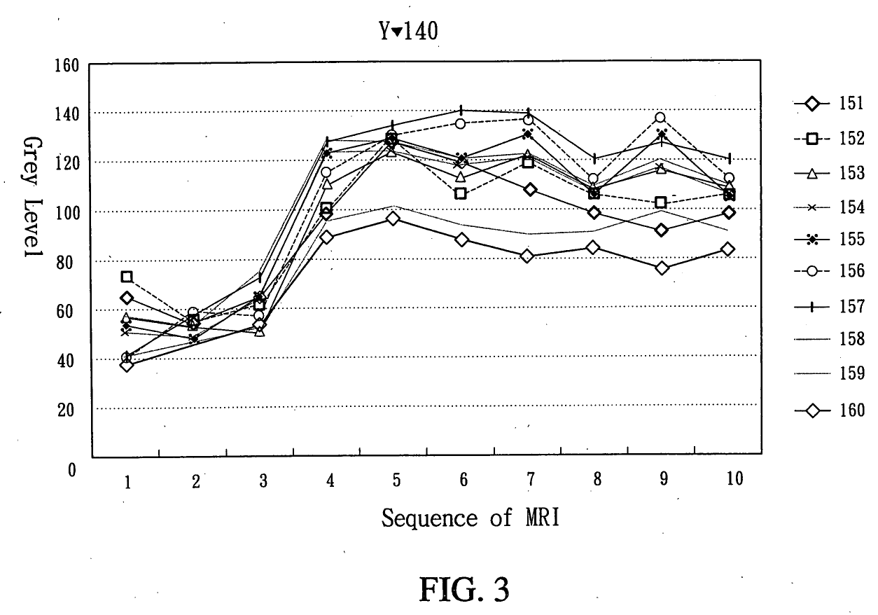

Grey prediction is used to locate the possible tumor regions for the rough segmentation. After this segmentation, the amount of computation is reduced and the precision of finding correct tumor area is increased dramatically. In this procedure, the segmentation is based on the traditional tumor detection assumption of dynamic MRI: the variations of intensities in tumor region are large than normal region increasingly. In real cases, the intensities may not change linearly and may decrease after specific time. FIG. 3 shows the intensities change of the tumor region through ten different time frames in dynamic MRI.

Although the intensities do not increase as the traditional tumor detection assumption, the rough segmentation of candidate tumor regions is well performed by grey prediction method. The developing coefficient and control coefficient are used to filter the candidate tumor regions roughly.
X⁽¹⁾(i)=[X⁽⁰⁾(1)−b/a]e^{-a(i-1)}+b/a (3)
X⁽⁰⁾(i)=[X⁽⁰⁾(1)−b/a](1−e^{a})e^{-a(i-1)} (4)
wherein X⁽¹⁾(i): the i^{th} after accumulated generating operation
X⁽⁰⁾(i): the i^{th} predict value of the original numerical series
X⁽⁰⁾(1): the first predict value of the original numerical series
a: developing coefficient
b: control coefficient

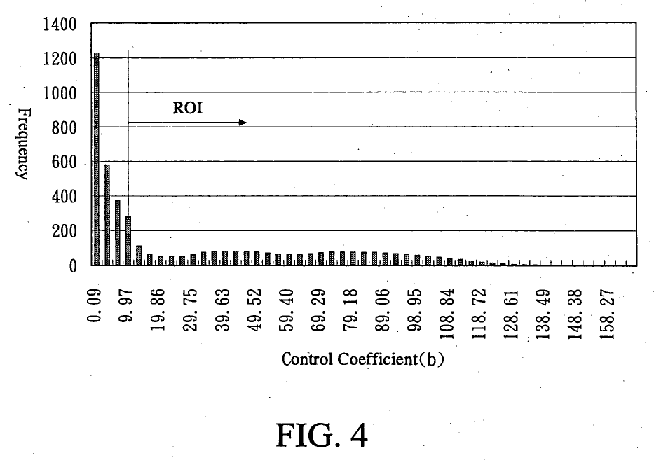

The intensities around the tumor region become bright more quickly than the normal region in dynamic MRI. According to equation (4), if a is positive, the predicted value will approach to zero gradually, no matter b is positive or negative. If a and b both are negative, the predicted value could be positive or negative. If a is negative and b is positive, then the predicted value will be on the increase. Therefore, in this research, those values of negative a are collected as the filtered values represented as those image points of increasing intensities. Based on these filtered image points, the probability distribution of b can be calculated. Then the threshold value of b located as the reflection point between the first top and valley after such a top of the wave. This threshold value represents control coefficient, which is greater than some value among those increasing-intensity image points. FIG. 3 shows the frequency distribution of control coefficient for those negative developing coefficient points. In FIG. 3, the reflection point between the first top and valley of wave is 9.975. Notice that the threshold value is computed automatically. FIG. 4 shows the rough segmentation of possible tumor area through the threshold.

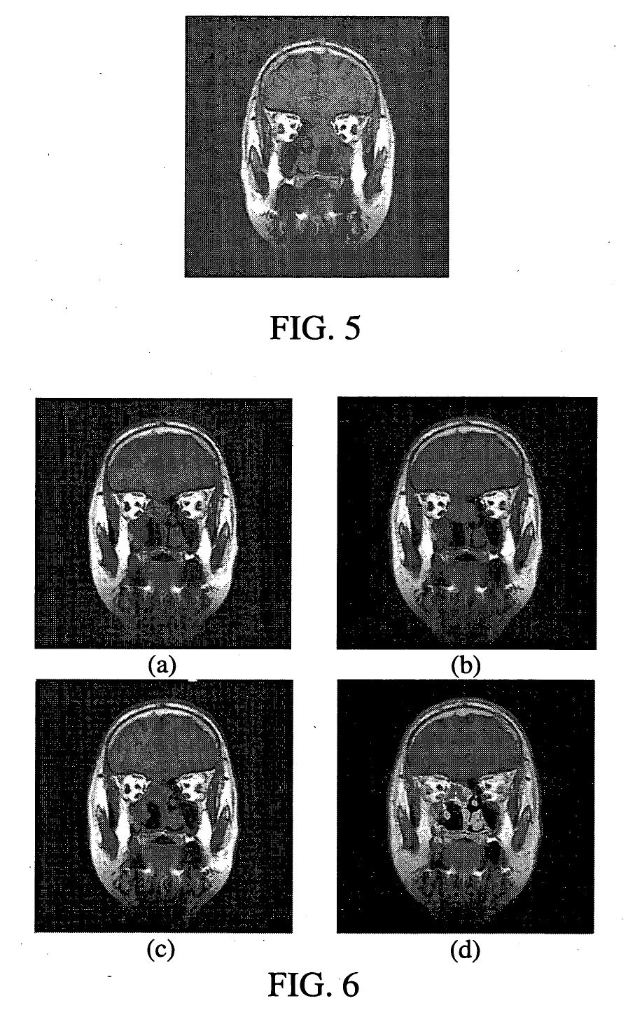

FIG. 5 shows the possible tumor region after rough segmentation. Comparing FIGS. 2 and 5, the candidate regions detected by grey prediction contain those image points whose intensities are increasing. Those image points with decreasing or unruly changed intensities are not selected by grey prediction. The rough segmentation reduces the amount of consequent data processing and increases the precision rate of correctly detecting tumor region.

(2b) Refinedly Segmenting the Images by Fuzzy C-means Clustering

After the rough process by grey prediction, Fuzzy C-means Clustering (FCM) is used for refined segmentation between tumor and normal regions. FCM partitions a collection of n vector Vᵢ, i=1, . . , n into G fuzzy groups, and finds a cluster center in each group such that a cost function of dissimilarity measure is minimized. Detailed algorithm is described in. Let G, the number of fuzzy groups, be two. One group is for the normal region and the other is for the tumor region.

FIG. 6 shows the results after performing the proposed automatic tumor detection algorithm after grey prediction and FCM clustering. FIG. 6(a) shows one of the original images. FIG. 6(b) shows the manual target tumor region identified by the radiologist. FIG. 6(c) shows the result after grey prediction; the red color indicates the candidate tumor areas. FIG. 6(d) shows the results performed by FCM clustering algorithm based on the result after grey prediction. The green area in FIG. 6(d) is the filtered area after FCM clustering.

As illustrated in the above preferred embodiment, a fully automatic nasal tumor detection system has been developed for dynamic MR images. The algorithm has already been examined on different MR image sequences. Most of the results are robust and correct. However, as the future work, the parameters selection in grey prediction stage should be optimally assigned for the best performance.

What is claimed is:

1. A method for automatically detecting a nasal tumor, comprising steps of:
(a) roughly segmenting at least two MR (magnetic resonance) images by grey prediction to locate candidate tumor regions; and
(b) refinedly segmenting said MR images of step (a) by Fuzzy C-means clustering to filter a possible tumor region from normal regions.

2. The method as claimed in claim 1, wherein said MR images of step (a) are previously transformed into a grey level format.

3. The method as claimed in claim 2, wherein said MR images have a width of 256 pixels and a height of 256 pixels.

4. The method as claimed in claim 2, wherein said MR images are transformed into images without header information.

5. The method as claimed in claim 2, wherein said images are transformed into 256 grey levels in each pixel thereof.

6. The method as claimed in claim 1, wherein corresponding points of said MR images are previously matched with each other.

7. The method as claimed in claim 6, wherein said corresponding points of said images are matched by a phase correlation process and a function minimization process.

8. The method as claimed in claim 1, wherein said images are segmented in step (a) according to developing coefficient and control coefficient of grey prediction.

9. A method for automatically detecting a nasal tumor, comprising steps of:
(1a) transforming at least two MR (magnetic resonance) images into a grey level format;
(1b) matching corresponding points of said MR images with each other;
(2a) roughly segmenting said MR images by grey prediction to locate candidate tumor regions; and
(2b) refinedly segmenting said MR images of step (2a) by Fuzzy C-means clustering to filter a possible tumor region from normal regions.

10. The method as claimed in claim 9, wherein said MR images of step (1a) have a width of 256 pixels and a height of 256 pixels.

11. The method as claimed in claim 9, wherein said MR images of step (1a) are transformed into images without header information.

12. The method as claimed in claim 9, wherein said images of step (1a) are transformed into 256 grey levels in each pixel thereof.

13. The method as claimed in claim 9, wherein said corresponding points of said images of step (1b) are matched by a phase correlation process and a function minimization process.

14. The method as claimed in claim 9, wherein said images of step (2a) are segmented according to developing coefficient and control coefficient of grey prediction.
